(19) United States
(12) Patent Application Publication
(10) Pub. No.: US 2022/0000414 A1
(43) Pub. Date: Jan. 6, 2022

(54) SYSTEMS AND METHODS FOR DETECTING COGNITIVE DISEASES AND IMPAIRMENTS IN HUMANS

(71) Applicant: The Research Foundation for the State University of New York, Albany, NY (US)

(72) Inventors: Igor Lednev, Glenmont, NY (US); Nicole Ralbovsky, Niskayuna, NY (US)

(21) Appl. No.: 17/368,251

(22) Filed: Jul. 6, 2021

Related U.S. Application Data
(60) Provisional application No. 63/047,976, filed on Jul. 3, 2020.

(51) Int. Cl. A61B 5/00; A61B 10/00

(52) U.S. Cl. CPC A61B 5/4088; A61B 10/0051; A61B 5/7246; A61B 5/0075

(57) ABSTRACT

Systems and methods for detecting a cognitive diseases and/or impairments in humans are disclosed. The method may include providing a saliva sample from a human subject, and subjecting at least a portion of the saliva sample to a spectroscopic analysis to produce a sample spectroscopic signature. The method may also include analyzing the produced sample spectroscopic signature using a predetermined statistical model. The predetermined statistical model may be based on spectroscopic signatures for a plurality modeling samples, and the spectroscopic signatures for each of the plurality of modeling samples may be associated with one of a plurality of predetermined cognitive categories. Additionally, the method may include correlating the produced sample spectroscopic signature with one of the plurality of predetermined cognitive categories based on the spectroscopic signatures for each of the plurality of modeling samples of the predetermined statistical model.

### CROSS-REFERENCE TO RELATED APPLICATIONS

This application claims priority to U.S. provisional application No. 63/047,976 filed on Jul. 3, 2020, the content of which is hereby incorporated by reference into the present application

### BACKGROUND

The disclosure relates generally to methods of detecting human diseases and impairments, and more particularly, to methods for detecting cognitive disease and impairments in humans including Alzheimer's disease (AD) and/or mild cognitive impairment (MCI) conditions.

Current diagnostic efforts for Alzheimer's disease involve a lengthy and arduous combination of tests and examinations. Variations of mental health and mood exams as well as physical, neurological, and imaging tests are among those used. Although a combination of these tests can lead to a “loose” diagnosis, there are considerable drawbacks. Cerebrospinal fluid analysis is invasive, requiring a lumbar puncture. The Mini-Mental State Examination, although scored, can be fairly subjective. Imaging tests are expensive and work only to eliminate other potential diseases.

### BRIEF DESCRIPTION

A first aspect of the disclosure provides a method for detecting a cognitive disease. The method includes: providing a saliva sample from a human subject; subjecting at least a portion of the saliva sample to a spectroscopic analysis to produce a sample spectroscopic signature for the saliva sample; analyzing the produced sample spectroscopic signature using a predetermined statistical model, the predetermined statistical model based on spectroscopic signatures for a plurality modeling samples, wherein the spectroscopic signatures for each of the plurality of modeling samples are associated with one of a plurality of predetermined cognitive categories; and correlating the produced sample spectroscopic signature with one of the plurality of predetermined cognitive categories based on the spectroscopic signatures for each of the plurality of modeling samples of the predetermined statistical model.

A second aspect of the disclosure provides a system including: a spectroscopy device subjecting at least a portion of a saliva sample from a human to a spectroscopic analysis to produce a sample spectroscopic signature for the saliva sample; and at least one computing device in operable communication with the spectroscopy device, the at least one computing device configured to detect a cognitive disease in the human subject by: analyzing the produced sample spectroscopic signature using a predetermined statistical model, the predetermined statistical model based on spectroscopic signatures for a plurality modeling samples, wherein the spectroscopic signatures for each of the plurality of modeling samples are associated with one of a plurality of predetermined cognitive categories; and correlating the produced sample spectroscopic signature with one of the plurality of predetermined cognitive categories based on the spectroscopic signatures for each of the plurality of modeling samples of the predetermined statistical model.

The illustrative aspects of the present disclosure are designed to solve the problems herein described and/or other problems not discussed.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 shows a schematic view of a system for detecting cognitive diseases and impairments in humans, according to embodiments of the disclosure.

FIG. 2A shows a spectroscopic signature produced from a saliva sample from a human that has no cognitive diseases and/or impairments, according to embodiments of the disclosure.

FIG. 2B shows a spectroscopic signature produced from a saliva sample from a human that has mild cognitive impairment (MCI), according to embodiments of the disclosure.

FIG. 2C shows a spectroscopic signature produced from a saliva sample from a human that has Alzheimer's disease (AD), according to embodiments of the disclosure.

FIG. 3 shows the various spectroscopic signatures shown in FIGS. 2A-2C overlaying one another, according to embodiments of the disclosure.

FIG. 4 shows a spectroscopic signature produced from a saliva sample from a human that is used to determine if the human has a cognitive disease or impairment, according to embodiments of the disclosure.

FIG. 5 shows the spectroscopic signature of FIG. 4 overlaid over each of the spectroscopic signatures shown in FIG. 3, according to embodiments of the disclosure.

FIG. 6 shows the spectroscopic signature of FIG. 4 overlaid over the spectroscopic signatures shown in FIG. 2C, according to embodiments of the disclosure.

FIG. 7 show various flowcharts illustrating processes for detecting cognitive diseases and impairments in a human, according to embodiments of the disclosure.

FIG. 8 shows a schematic view of a computing system configured to detecting cognitive diseases and impairments in humans, according to embodiments of the disclosure.

It is noted that the drawings of the disclosure are not to scale. The drawings are intended to depict only typical aspects of the disclosure, and therefore should not be considered as limiting the scope of the disclosure. In the drawings, like numbering represents like elements between the drawings.

### DETAILED DESCRIPTION

As an initial matter, in order to clearly describe the current disclosure it will become necessary to select certain terminology when referring to and describing relevant components within the disclosure. When doing this, if possible, common industry terminology will be used and employed in a manner consistent with its accepted meaning. Unless otherwise stated, such terminology should be given a broad interpretation consistent with the context of the present application and the scope of the appended claims. Those of ordinary skill in the art will appreciate that often a particular component may be referred to using several different or overlapping terms. What may be described herein as being a single part may include and be referenced in another context as consisting of multiple components. Alternatively, what may be described herein as including multiple components may be referred to elsewhere as a single part.

As discussed herein, the disclosure relates generally to methods of detecting human diseases and impairments, and more particularly, to methods for detecting cognitive disease and impairments in humans including Alzheimer's disease and/or mild cognitive impairment (MCI) conditions.

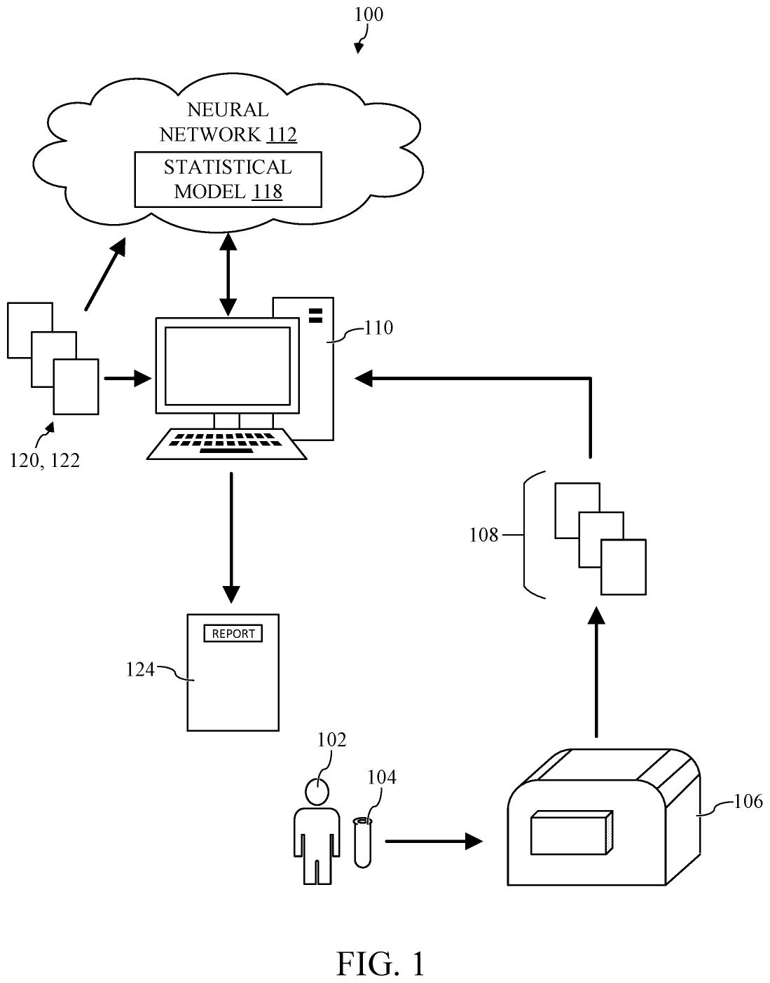

These and other embodiments are discussed below with reference to FIGS. 1-8. However, those skilled in the art will readily appreciate that the detailed description given herein with respect to these Figures is for explanatory purposes only and should not be construed as limiting.

FIG. 1 shows a schematic view have a system 100 for detecting cognitive diseases. More specifically, FIG. 1 shows a non-limiting example of system 100 that may detect cognitive diseases and/or mental impairments in humans/human subjects 102 using saliva samples 104. As discussed herein, cognitive diseases and/or mental impairments that may be detected in humans 102 using system 100 may include Alzheimer's Diseases (AD) or mental cognitive impairment (MCI)—which may be a precursor to AD.

System 100 shown in FIG. 1 may include a spectroscopy device 106 configured to analyze saliva samples 104. That is, system 100 may include spectroscopy device 106 configured to subject at least a portion of a saliva sample 104 from a human 102 to a spectroscopic analysis to produce a sample spectroscopic signature 108 for the saliva sample 104. In one limiting example, only a single portion of saliva sample 104 may be analyzed, processed, and/or examined by spectroscopy device 106 to generate a single sample spectroscopic signature 108 for additional processing by system 100. In another non-limiting example, a plurality of portions or substantially the entirety of sample 104 from human 106 may be analyzed, processed, and/or examined by spectroscopy device 106 to generate a plurality of sample spectroscopic signatures 108 for further processing by system 100. That is, a plurality of portions of saliva sample 104 may be subject to spectroscopic analysis by spectroscopy device 106 to produce a plurality of distinct sample spectroscopic signatures 108 for saliva sample 104. Each of the plurality of portions of saliva sample 104 undergoing the spectroscopic analysis may be positionally distinct from the others in saliva sample 104.

In a non-limiting example, the spectroscopic analysis performed on saliva sample 104 using spectroscopic device 106 may include performing Raman spectroscopy. The Raman spectroscopy process may include, but may not be limited to: near-infrared (NIR) Raman spectroscopy, Raman microspectroscopy, Surface Enhanced Raman spectroscopy (SERS), surface enhanced resonance Raman spectroscopy (SERRS), Raman hyper spectroscopy, Fourier transform Raman spectroscopy, and coherent anti-Stokes Raman Spectroscopy (CARS).

Raman spectroscopy is a spectroscopic technique which relies on inelastic or Raman scattering of monochromatic light to study vibrational, rotational, and other low-frequency modes in a system (Gardiner, D. J., Practical Raman Spectroscopy, Berlin: Springer-Verlag, pp. 1-3 (1989), which is hereby incorporated by reference in its entirety). Vibrational modes are very important and very specific for certain types of chemical groups in molecules. They provide a “fingerprint” by which a molecule or biomolecule can be identified. The Raman effect is obtained when a photon interacts with the electron cloud of a molecule, exciting the electrons into a virtual state. The scattered photon is shifted to lower frequencies (Stokes process) or higher frequencies (anti-Stokes process) as it releases energy to or from the molecule, respectively. The polarizability change in the molecule will determine the Raman scattering efficiency, while the Raman shift will be equal to the energy (frequency) of the vibrational mode involved.

Fluorescence interference is the largest problem with Raman spectroscopy and is perhaps the reason why the latter technique has not been more popular in the past. If a sample contains molecules that fluoresce, the broad and much more intense fluorescence peak will mask the sharp Raman peaks of the sample. There are a few remedies to this problem. One solution is to use deep ultraviolet (DUV) light for exciting Raman scattering (Lednev I. K., “Vibrational Spectroscopy: Biological Applications of Ultraviolet Raman Spectroscopy,” in: V. N. Uversky, and E. A. Permyakov, Protein Structures, Methods in Protein Structures and Stability Analysis (2007), which is hereby incorporated by reference in its entirety). Practically no condensed face exhibits fluorescence below ^{˜}250 nm. Possible photodegradation of biological samples is an expected disadvantage of DUV Raman spectroscopy. Another option to eliminate fluorescence interference is to use a near-IR (NIR) excitation for Raman spectroscopic measurement. Finally, surface enhanced Raman spectroscopy (SERS) which involves a rough metal surface can also alleviate the problem of fluorescence (Thomas et al., “Raman Spectroscopy and the Forensic Analysis of Black/Grey and Blue Cotton Fibers Part 1: Investigation of the Effects of Varying Laser Wavelength,” Forensic Sci. Int. 152:189-197 (2005), which is hereby incorporated by reference in its entirety). However, this method requires direct contact with the analyte and cannot be considered to be nondestructive.

Basic components of a Raman spectrometer are (i) an excitation source; (ii) optics for sample illumination; (iii) a single, double, or triple monochromator; and (iv) a signal processing system consisting of a detector, an amplifier, and an output device.

Typically, a sample is exposed to a monochromatic source usually a laser in the visible, near infrared, or near ultraviolet range. The scattered light is collected using a lens and is focused at the entrance slit of a monochromator. The monochromator, which is set for a desirable spectral resolution rejects the stray light in addition to dispersing incoming radiation. The light leaving the exit slit of the monochromator is collected and focused on a detector (such as a photodiode arrays (PDA), a photomultiplier (PMT), or charge-coupled device (CCD)). This optical signal is converted to an electrical signal within the detector. The incident signal is stored in computer memory for each predetermined frequency interval. A plot of the signal intensity as a function of its frequency difference (usually in units of wavenumbers, cm−1) will constitute the Raman spectroscopic signature (e.g., sample spectroscopic signature 108).

Raman signatures are sharp and narrow peaks observed on a Raman spectrum. These peaks are located on both sides of the excitation laser line (Stoke and anti-Stoke lines). Generally, only the Stokes region is used for comparison (the anti-Stoke region is identical in pattern, but much less intense) with a Raman spectrum of a known sample. A visual comparison of these set of peaks (spectroscopic signatures) between experimental and known samples is needed to verify the reproducibility of the data. Therefore, establishing correlations between experimental and known data is required to assign the peaks in the molecules and identify a specific component in the sample.

In another non-limiting example, vibrational spectroscopy may be used in system 100. In the non-limiting example, vibrational spectroscopy may include or involve Infrared (IR) absorption, Fourier Transform Infrared absorption (FTIR), Attenuated Total Reflection (ATR) FTIR or IR reflection spectroscopy. In this example where vibrational spectroscopy may be used, generated sample spectroscopic signature 108 may include a vibrational signature of saliva sample 104.

Fourier transform infrared (FTIR) spectroscopy is a versatile tool for the detection and structural determination of organic and inorganic compounds. In infrared spectroscopy (IR), IR radiation is passed through a sample. Some of the infrared radiation is absorbed by the sample and some is transmitted. The resulting spectrum represents a fingerprint of a sample with absorption peaks which correspond to the frequencies of vibrational modes of the material. Basic components of FTIR are (i) a light source; (ii) an interferometer; (iii) a sample; (iv) a detector; (v) computer.

Infrared radiation is emitted from a glowing black-body source. This beam passes through an aperture which controls the amount of energy presented to the sample (and, ultimately, to the detector). The beam enters the interferometer where “spectral encoding” takes place. The resulting interferogram signal then exits the interferometer. The beam enters the sample compartment where it is transmitted through or reflected off of the surface of the sample; depending on the type of analysis being accomplished, this is where specific frequencies, which are uniquely characteristic of the sample, are absorbed. The beam finally passes to the detector for final measurement. The measured signal is digitized and sent to the computer where the Fourier transformation takes place.

Microscopic-attenuated total reflectance (ATR) Fourier transform infrared (FT-IR) analysis is nondestructive, with analysis times competitive to current methodologies, and offers a molecular or biomolecule “fingerprint” of the analyzed sample. The vibrational signatures collected from the sample are easily discernible by the naked eye. Furthermore, this vibrational “fingerprint” targets a wider range of chemicals as compared to current methodologies, increasing the selectivity of the method. The optics of ATR-FT-IR imaging provides pseudo-immersion analysis. The high refractive index of the germanium ATR crystal increases the numerical aperture of the optics, enhancing spatial resolution by a factor of 4, without the use of a synchrotron light source (ATR accessories, An overview, PerkinElmer Life and Analytical Sciences, (2004), which is hereby incorporated by reference in its entirety). Exhaustive research applying micro-ATR-FT-IR chemical imaging (mapping) to the fields of bio-medical (Chan et al., Appl. Spectrosc. 59:149 (2005); Anastassopoulou et al., Vibrational Spectroscopy, 51:270 (2009); Kazarian et al., Biochimica et Biophysica Acta (BBA)—Biomembranes, 1758:858 (2006); Kazarian et al., Analyst, 138:1940 (2013), which are hereby incorporated by reference in their entirety) and forensic research (Dirwono et al., Forensic Science International, 199:6 (2010); Ng et al., Anal. and Bioanal. Chem. 394:2039 (2009); Spring et al., Anal. and Bioanal. Chem., 392:37 (2008), which are hereby incorporated by reference in their entirety) have been reported.

As discussed herein, subjecting saliva sample 104 to spectroscopic analysis via spectroscopic device 106 to generate sample spectroscopic signatures 108 may include for example, exposing biomolecules of saliva sample 104 to a spectroscopic analysis generated/performed by spectroscopy device 106. The exposure of biomolecules may, at least in part, contribute to the generation of sample spectroscopic signatures 108 and/or form the “fingerprinting” type of information included in produced signatures 108, as discussed herein. For example, at least one of the structural properties, conformational properties, or compositional variations of the exposed biomolecules may define the produced sample spectroscopic signature 108 for each saliva sample 104 analyzed by spectroscopy device 106. In non-limiting examples, the biomolecules that may aid in the generation and/or production of spectroscopic signature 108 may include, but are not limited to, proteins, lipids, peptides, amino acids, electrolytes, mucus, enzymes, and/or antibacterial species.

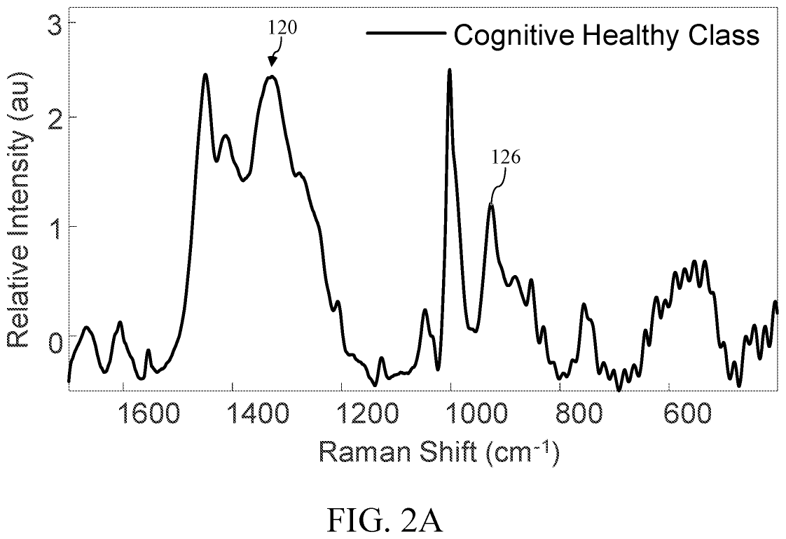

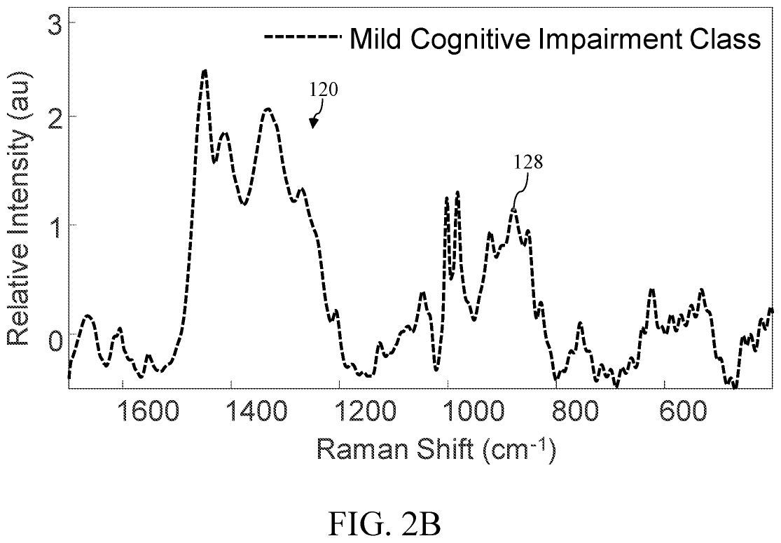

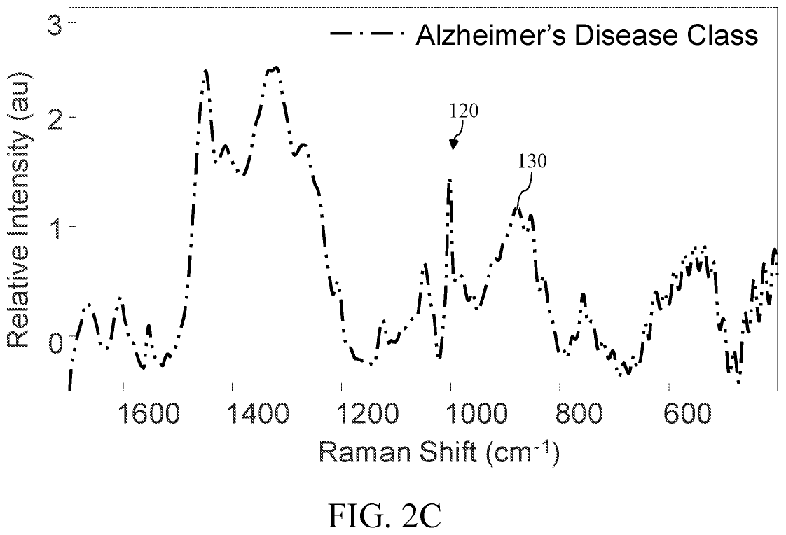

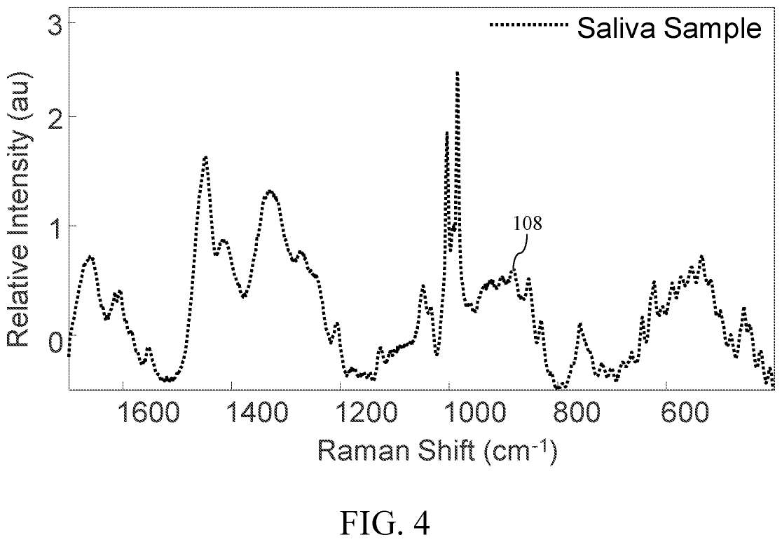

In non-limiting examples discussed herein (see, FIGS. 2A-6), sample spectroscopic signature(s) 108 may include and/or provide a plurality of data or information. That is, the data, for example plot points, included in each sample spectroscopic signature 108 produced or generated as a result of performing spectroscopy analysis on saliva sample 104 may aid in the detection of cognitive diseases and/or mental impairments in humans 102. In a non-limiting example where only a single portion of saliva sample 104 is analyzed, only a single set of spectroscopic signature 108 data points or information may be generated or produced (e.g., a single graph worth of plot points—see FIG. 4). In other non-limiting examples where a plurality of distinct portions of saliva sample 104 are analyzed using spectroscopy device 106, multiple, distinct sets of spectroscopic signature 108 or information may be generated or produced (e.g., a plurality of distinct graphs worth of plot points). As discussed herein, the multiple distinct sets of spectroscopic signature 108 data points may be processed separately to detect cognitive diseases/impairments in human 102, or alternatively may be averaged to form a single set of data points, prior to processing.

As shown in FIG. 1, system 100 may also include at least one computing device 110. Computing device 110 may be in operable communication with spectroscopy device 106. More specifically, computing device 110 may be connected to, in communication with, and/or operably connected with spectroscopy device 106. As a result, and during operation, computing device 110 may receive spectroscopic signature(s) 108 generated or produced by spectroscopy device 106 and may perform processes an spectroscopic signature(s) 108 to detect cognitive diseases and/or mental impairments, as discussed herein. Computing device 110 may be a stand-alone device, or alternatively may be a portion and/or included in a larger computing device (not shown) of system 100. For example, and as shown in FIG. 1, computing device 110 may be separate from spectroscopy device 106. Alternatively, computing device 110 may be part of the overall computing system that is used in the operation of spectroscopy device 106. As such, computing device 110 may be formed as any device and/or computing system/network that may be configured to perform the processes discussed herein to identify or detect cognitive diseases/impairments in humans. As discussed herein, computing device 110 may be configured to process spectroscopic signature(s) 108 to detect cognitive diseases or impairments using spectroscopic signature(s) 108. As shown in FIG. 1, computing device 110 may be in electronic communication with and/or communicatively coupled to various devices, apparatuses, and/or portions of system 100. In non-limiting examples, computing device 110 may be hard-wired and/or wirelessly connected to and/or in communication with spectroscopy device 106, and/or other components via any suitable electronic and/or mechanical communication component or technique. For example, computing device 110 may be in electronic communication with spectroscopy device 106 and neural network 112. Additionally, and as discussed herein, computing device 110 may also receive, process, and/or analyze spectroscopic signature(s) 108 during the processes discussed herein.

System 100 may also include a neural network 112. In the non-limiting example shown in FIG. 1, neural network 112 may be distinct/separate from and in communication and/or operably coupled to computing device 110. In another non-limiting example, neural network 112 may be included within and/or formed as a part of computing device 110. Neural network 112 may be any suitable component, device, program product, and/or system that may be configured to aid in the process of detecting cognitive diseases or mental impairments in humans based on spectroscopic signature(s) 108, as discussed herein. For example, neural network 112 may be formed as any suitable machine learning device, program, and/or series of algorithms including, but not limited to, artificial/simulated neural networks including a plurality of interconnected, hidden layer nodes.

Upon receiving the plurality of spectroscopic signature(s) 108 generated/captured by spectroscopy device 106, computing device 110 using neural network 112 may perform a plurality of processes, manipulations and/or calculations using spectroscopic signature(s) 108 to detect cognitive diseases or mental impairments in humans. In a non-limiting example shown in FIG. 1, neural network 112 may utilize a predetermined statistical model 118 to process spectroscopic signature(s) 108 in order to detect cognitive diseases/impairments. That is, statistical model 118 included within neural network 112 may be used to analyze produced sample spectroscopic signature(s) 108. The predetermined statistical model 118 may be based on spectroscopic signatures 120 for a plurality modeling samples 122. In this example, spectroscopic signatures 120 for each of the plurality of modeling samples 122 are associated with one of a plurality of predetermined cognitive categories. More specifically, predetermined statistical model 118 may be built, generated, created, established, and/or based on spectroscopic signatures 120 for each of the plurality of modeling samples 122 associated with predetermined cognitive categories. In a non-limiting example, the predetermined cognitive categories may include (1) a cognitive healthy class, (2) an Alzheimer's disease class, and (3) a mild cognitive impairment class. As such, each of the predetermined spectroscopic signatures 120/modeling samples 122 provided to and/or used by neural network 112 to build predetermined statistical model 118 may have a known associated cognitive category prior to being used by neural network 112 to form statistical model 118.

In the example, after analyzing the produced sample spectroscopic signature(s) 108 using predetermined statistical model 118, sample spectroscopic signature(s) 108 may be correlated with one of the plurality of predetermined cognitive categories. Sample spectroscopic signature(s) 108 may be correlated with one of the plurality of predetermined cognitive categories based on the spectroscopic signatures 120 for each of the plurality of modeling samples 122 of the predetermined statistical model 118. That is, using statistical model 118, which may be generated based on spectroscopic signatures 120 for the plurality of modeling samples 122, produced sample spectroscopic signature(s) 108 may be correlated, associated, linked, and/or related to one of the predetermined cognitive categories. Computing device 110 using statistical model 118 may correlate sample spectroscopic signature(s) 108 by identifying human subject 102 as being associated with one of (1) the cognitive healthy class, (2) the Alzheimer's disease class, or (3) the mild cognitive impairment class. Once identified as being associated with one of the predetermined cognitive categories, computing device 110 may subsequently detect a cognitive disease or mental impairment in human 102 in response to identifying human subject 102 being associated with one of the Alzheimer's disease class or the mild cognitive impairment class.

In another non-limiting example, each predetermined spectroscopic signatures 120/modeling samples 122 may have an associated age or age range. As such, produced spectroscopic signature(s) 108 provided to computing device 110 may also include a known age for human 102. This in turn may increase the accuracy and/or efficiency in detecting cognitive diseases and/or mental impairments in human 102 providing saliva sample 104.

Although shown as being part of neural network 112, it is understood that predetermined statistical model 118 may also or alternatively be stored or included in computing device 110. That is, neural network 112 may generate predetermined statistical model 118 and subsequently provide statistical model 118 to computing device 110, such that statistical model 118 may be run or operate directly on computing device 110. Predetermined statistical model 118 may be generated and/or created using any suitable statistical function, operation, and/or algorithm. For example, predetermined statistical model 118 may generated and/or created using Principle component analysis (PCA), Partial Least Squares Discriminant Analysis (PLSDA), Multilayer perceptrons (MLP), Radial Basis Function (RBF), artificial neural network (ANN), support vector machine (SVM), SVM-based Discriminant analysis, or the like.

Additionally, computing device 110 may generate a report 124 related to the findings, analysis, and/or potential detection of cognitive disease/mental impairment. In non-limiting examples, report 124 may be a physical print out, a graphical depiction provided on a display device of computing device 110 (e.g., screen monitor), or any other suitable visual representation providing information or data relating to the analysis of spectroscopic signature(s) 108 and/or the detection of cognitive diseases or mental impairments in human 102 providing saliva sample 104. As discussed herein, the generated report 124 may include visual information relating to the data points included, collected, and/or generated in spectroscopic signature(s) 108. Additionally, report 124 may include visual information relating the classification/correlation of the produced sample spectroscopic signature(s) 108 with one of the plurality of predetermined cognitive categories (e.g., (1) a cognitive healthy class, (2) an Alzheimer's disease class, and (3) a mild cognitive impairment class) based on spectroscopic signatures 120 for each of the plurality of modeling samples 122 of predetermined statistical model 118.

As discussed herein, report 124 generated by the processes performed by computing device 110 and neural network 112 may aid in a physician's ability to detect cognitive diseases or mental impairments in humans more quickly, more accurately, in early stages of the diseases/impairment, and/or less invasively than conventional processes.

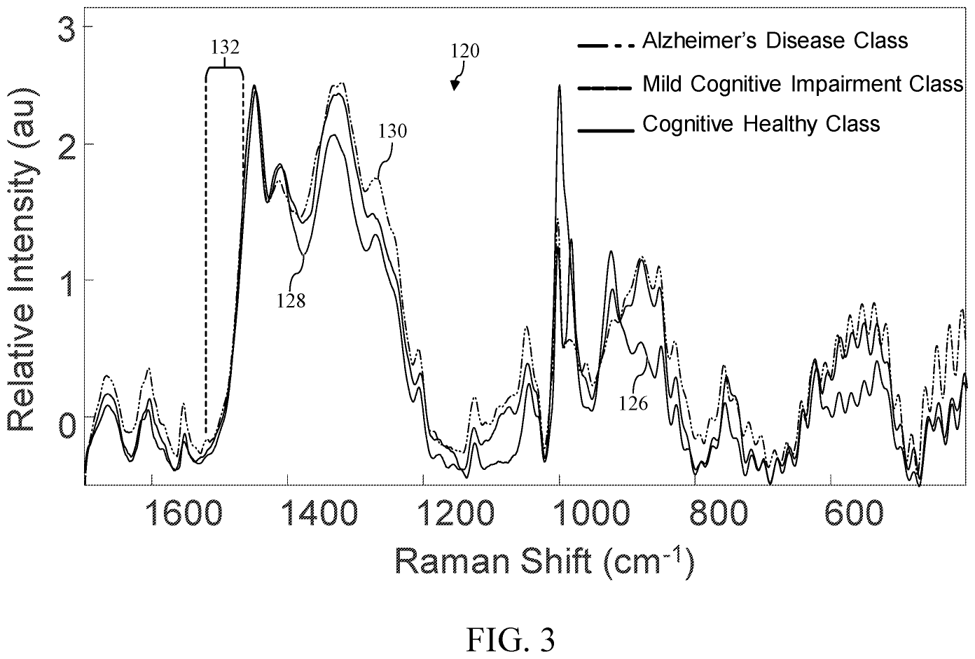

FIGS. 2A-3 show various views of spectroscopic signatures 120 for distinct modeling samples 122. More specifically, FIG. 2A shows a graphical representation of a spectroscopic signature 120, 126 produced from a saliva sample from a human (e.g., modeling sample 122) that has no cognitive diseases and/or impairments and is therefore classified as part of the cognitive healthy class. FIG. 2B shows a graphical representation of a spectroscopic signature 120, 128 produced from a saliva sample from a human (e.g., modeling sample 122) that that has mild cognitive impairment (MCI) and is therefore classified or identified as part of the mild cognitive impairment class. FIG. 2C shows a spectroscopic signature 120, 130 produced from a saliva sample from a human (e.g., modeling sample 122) that has Alzheimer's disease (AD), and is thus classified as part of the Alzheimer disease class. Finally, FIG. 3 shows the various spectroscopic signatures 120, 126, 128, 130 shown in FIGS. 2A-2C overlaying one another. Although three are shown, it is understood that a plurality of spectroscopic signatures 120, 126, 128, 130 from a plurality of modeling samples 122 may be used by computing device 110 and/or neural network 112 to generate or create predetermined statistical model 118. The ones shown are merely descriptive and/or illustrative.

Turning to FIG. 3, computing device 110 and/or neural network 112 may also remove portions of spectroscopic signatures 120, 126, 128, 130 of modeling samples 122 that may not be need and/or required to generate statistical model 118. For example, identified or predetermined portions 132 of each spectroscopic signature 126, 128, 130 may be determined by computing device 110/neural network 112 to be substantially similar and/or identical. As a result portions 132 may provide no indication of a difference in spectroscopic signature 126, 128, 130 between the various predetermined cognitive categories, and therefore may not aid in making a determination or distinction between spectroscopic signatures 126, 128, 130 for each of the categories. As such, computing device 110/neural network 112 may not analysis, compute, calculate, and/or examine portion 132 of spectroscopic signature 126, 128, 130 when generating, creating, and/or producing statistical model 118. Additionally, predetermined portion 132 may be discarded from the analysis, computation, calculation, and/or examination of spectroscopic signature 108, as discussed herein.

FIG. 4 shows a graphical representation of a spectroscopic signature 108 produced from saliva sample 104 from human 102 using system 100/spectroscopy device 106, as shown in FIG. 1. That is, FIG. 4 may represent the plotted graph or data points of spectroscopic signature 108 that may be generated by the spectroscopic analysis performed by spectroscopy device 106 on saliva sample 104. As shown, spectroscopic signature 108 produced from saliva sample 104 from human 102 may not be analyzed and/or correlated by computing device 110/neural network 112/statistical model 118. As such, produced sample spectroscopic signature 108 from saliva sample 104 may not yet be correlated to a predetermined cognitive category, and therefore, it may not yet be known whether human 102 has a cognitive disease/mental impairment, or not.

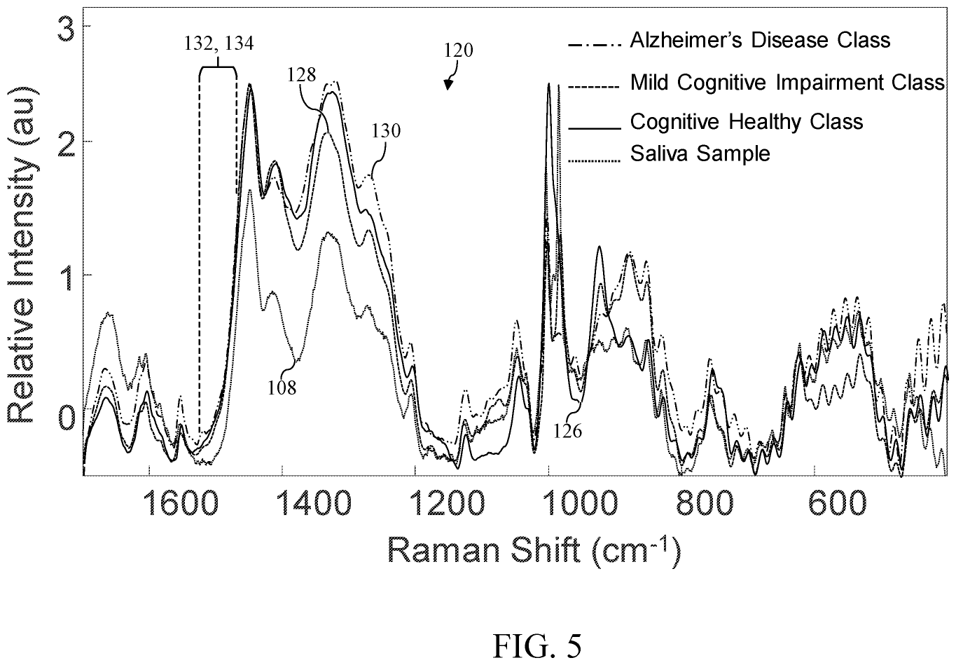

Turning to FIG. 5, spectroscopic signature 108 of saliva sample 104 may be overlaid spectroscopic signatures 120, 126, 128, 130 of modeling sample 122. This may indicate the providing of spectroscopic signature 108 of saliva sample 104 to computing device 110, neural network 112, and/or predetermined statistical model 118. Computing device 110, neural network 112, and/or predetermined statistical model 118 may analyze spectroscopic signature 108 of saliva sample 104 in view of statistical model 118. Analyzing spectroscopic signature 108 of saliva sample 104 may in turn may allow computing device 110, neural network 112, and/or predetermined statistical model 118 to correlate the produced sample spectroscopic signature 108 with one of the plurality of predetermined cognitive categories based on the spectroscopic signatures 120, 126, 128, 130 for each of the plurality of modeling samples 122 of predetermined statistical model 118.

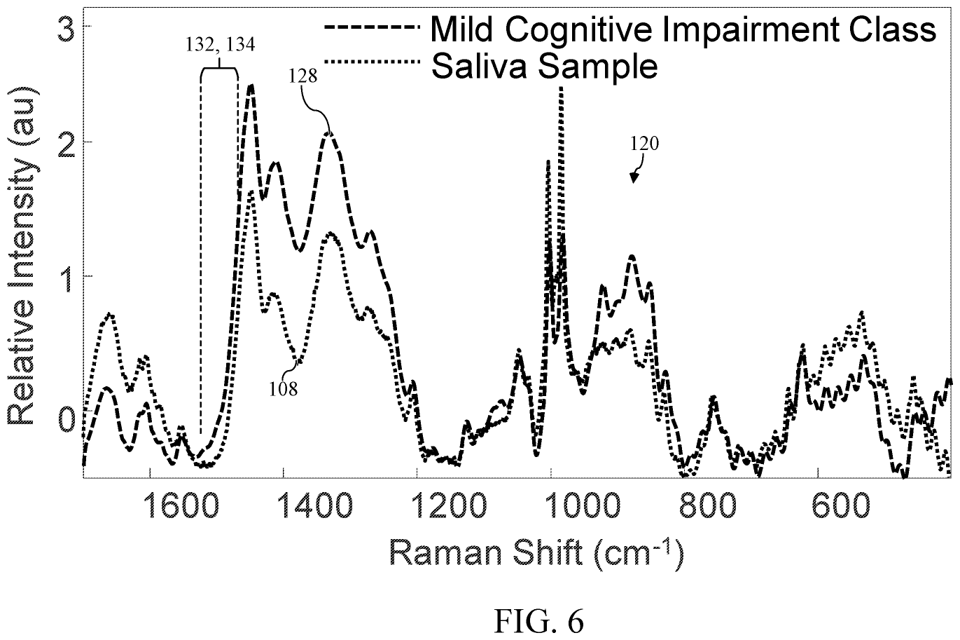

Turning to FIG. 6, computing device 110, neural network 112, and/or predetermined statistical model 118 may analyze spectroscopic signature 108 of sample 104 and determine that spectroscopic signature 108 correlates to, associates with , linked to, and/or related to a mild cognitive impairment class, as shown in spectroscopic signature 128 (see, FIG. 2B). That is, and based on statistical model 118, spectroscopic signature 108 of sample 104 may be correlated with spectroscopic signature 128 of modeling sample 122, which in turn may indicate, identify, determined, and/or detect that human 102 providing saliva sample 104 may have or suffer from a mild cognitive/mental impairment.

Also shown in FIG.6, computing device 110 and/or neural network 112 may also remove portions of spectroscopic signature 108 generated from saliva sample 104 that may not be needed and/or required to detect cognitive diseases and/or mental impairments in human 102. For example, and similarly to portion 132 of spectroscopic signatures 120, 126, 128, 130 discussed herein with respect to FIG. 3, identified or predetermined portions 134 of spectroscopic signature 108 may be determined by computing device 110/neural network 112 to be substantially similar, identical, and/or correspond to portion 132 of spectroscopic signatures 120, 126, 128, 130 of modeling samples 122. As a result, portions 134 may similarly provide no indication a cognitive disease/mental impairment and/or may not aid in the detection of the same. As such, computing device 110/neural network 112 may not analysis, compute, calculate, and/or examine predetermined portion 134 of spectroscopic signature 108 when analyzing or correlating spectroscopic signature 108 statistical model 118, as discussed herein. In a non-limiting example, predetermined portion 134 may be discarded from the analysis, computation, calculation, and/or examination of spectroscopic signature 108, as discussed herein.

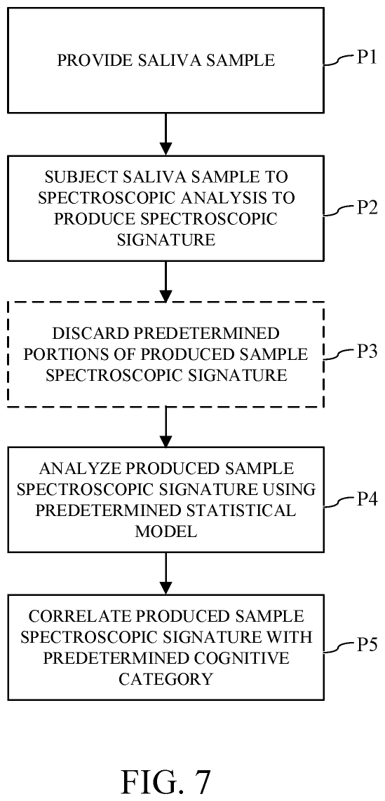

FIG. 7 depicts non-limiting example processes for detecting cognitive diseases. Specifically, FIG. 7 shows a flowchart depicting example processes for detecting cognitive diseases and/or mental impairments in humans. In some cases, a computing device(s) and/or system may be used to perform the processes for cognitive disease detection, as discussed herein with respect to FIG. 1.

Turning to FIG. 7, in process P1 a saliva sample may be provided. More specifically, a saliva sample from a human subject may be provided for analysis. The provided saliva sample may be provided for the purpose of detecting a cognitive disease and/or mental impairment in the human subject.

In process P2 the saliva sample may be subject to a spectroscopic analysis. More specifically, at least a portion of the saliva sample may be subject to a spectroscopic analysis to produce a sample spectroscopic signature for the saliva sample. Subjecting the saliva sample to the spectroscopic analysis may include performing Raman spectroscopy on at least a portion of the saliva sample. The Raman spectroscopy process or analysis performed on the saliva sample may include, but is not limit to, near-infrared (NIR) Raman spectroscopy, Raman microspectroscopy, Surface Enhanced Raman spectroscopy (SERS), surface enhanced resonance Raman spectroscopy (SERRS), Raman hyper spectroscopy, Fourier transform Raman spectroscopy, and coherent anti-Stokes Raman Spectroscopy (CARS). Subjecting the saliva sample to the spectroscopic analysis may include performing IR spectroscopy on at least a portion of the saliva sample. The IR spectroscopy process or analysis performed on the saliva sample may include, but is not limited to IR absorption spectroscopy, Fourier Transform Infrared absorption (FTIR), Attenuated Total Reflection (ATR) FTIR or IR reflection spectroscopy. Additionally, the subjecting of at least the portion of the saliva sample to the spectroscopic analysis may also include exposing biomolecules of the saliva sample to a spectroscopic analysis. The biomolecules may include structural properties, conformational properties, and/or compositional variations that define the produced sample spectroscopic signature for the saliva sample. In non-limiting examples, the biomolecules exposed to the spectroscopic analysis may include proteins, lipids, peptides, amino acids, electrolytes, mucus, enzymes, and/or antibacterial species.

In a non-limiting example, only one portion of saliva sample may be subject to spectroscopic analysis to generate or produce a single sample spectroscopic signature for the provided saliva sample. In another non-limiting example, a plurality of distinct portions of saliva sample may be subject to spectroscopic analysis in process P2. As such, a plurality of portions of the saliva sample may be subject to the spectroscopic analysis to produce a plurality of distinct sample spectroscopic signatures for the saliva sample. In this example each of the plurality of portions may be positionally distinct from the others in the saliva sample.

In process P3 (shown in phantom as optional), predetermined portions of the produced sample spectroscopic signature(s) may be discarded. That is, a predetermined portion of the produced sample spectroscopic signature(s) may be discarded prior to analyzing the produced sample spectroscopic signature(s) using the predetermined statistical model (see, process P4). The discarded, predetermined portions of the produced sample spectroscopic signature(s) may be associated with portions of the signature that are inconclusive for correlating the produced sample spectroscopic signature(s) with one of the plurality of predetermined cognitive categories.

In process P4, the produced sample spectroscopic signature may be analyzed. More specifically, the produced sample spectroscopic signature may be analyzed using a predetermined statistical model. The predetermined statistical model may be based on spectroscopic signature for a plurality of modeling samples. Additionally, the spectroscopic signatures for each of the plurality of modeling samples may be associated with one of a plurality of predetermined cognitive categories. In a non-limiting example, the plurality of predetermined cognitive categories may include: (1) a cognitive healthy class, (2) an Alzheimer's disease class, and (3) a mild cognitive impairment class.

In a non-limiting example where only a single spectroscopic signature is produced from the saliva sample, only the single produced spectroscopic signature may be analyzed. In other non-limiting examples where a plurality of spectroscopic signatures are produced from the saliva sample, analyzing in process P4 may include analyzing each of the plurality of the produced sample spectroscopic signatures using the predetermined statistical model.

In process P5, the produced sample spectroscopic signature may be correlated with one of the plurality of predetermined cognitive categories. More specifically, the produced sample spectroscopic signature may be correlated with one of the plurality of predetermined cognitive categories based on the spectroscopic signatures for each of the plurality of modeling samples of the predetermined statistical model. In a non-limiting example, correlating of the produced sample spectroscopic signature may also include identifying the human subject as being associated with one of the cognitive healthy class, the Alzheimer's disease class, or the mild cognitive impairment class. Additionally in the non-limiting example, correlating may include detecting the cognitive disease in the human subject in response to identifying the human subject being associated with one of the Alzheimer's disease class or the mild cognitive impairment class.

In a non-limiting example where a plurality of sample spectroscopic signatures are produced (e.g., process P2) and analyzed (e.g., process P4), correlating may include correlating each of the plurality of produced sample spectroscopic signatures with one of the plurality of predetermined cognitive categories based on the spectroscopic signatures for each of the plurality of modeling samples of the predetermined statistical model. Additionally in the non-limiting example, correlating each of the plurality of produced sample spectroscopic signatures may further include determining a final, predetermined cognitive category for the saliva sample based on each of the plurality of correlated, produced sample spectroscopic signatures.

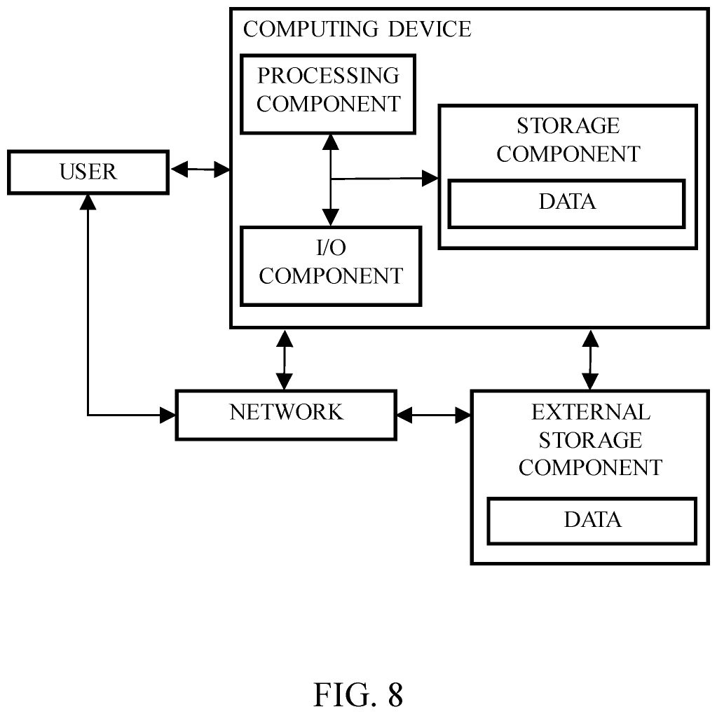

FIG. 8 depicts a schematic view of a computing environment or system (hereafter, “computing system”), and the various components included within computing system. In the non-limiting example shown in FIG. 8, computing system may include at least one computing device that may be configured to detect cognitive diseases and/or mental impairments in humans by performing the processes P1-P5 discussed herein with respect to FIG. 7. It is understood that similarly numbered and/or named components may function in a substantially similar fashion. Redundant explanation of these components has been omitted for clarity.

It is understood that computing device(s) may be implemented as a computer program product stored on a computer readable storage medium. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Python, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and/or computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Computing system shown in FIG. 8 may include any type of computing device(s) and for example includes at least one processor or processing component(s), storage component, input/output (I/O) component(s) (including a keyboard, touchscreen, or monitor display), and a communications pathway. In general, processing component(s) execute program code which is at least partially fixed or stored in storage component. While executing program code, processing component(s) can process data, which can result in reading and/or writing transformed data from/to storage component and/or I/O component(s) for further processing. The pathway provides a communications link between each of the components in computing device(s). I/O component can comprise one or more human I/O devices, which enables user to interact with computing device(s) to analyze produced sample spectroscopic signatures and detect cognitive diseases and/or mental impairments in humans, as discussed herein. Computing device(s) may also be implemented in a distributed manner such that different components reside in different physical locations.

Storage component may also include modules, data and/or electronic information relating to various other aspects of computing system. Specifically, operational modules, electronic information, and/or data relating to saliva sample data, spectroscopic analysis (e.g., Raman spectroscopy) data, spectroscopic signature data, predetermined statistical model data, predetermined cognitive categories data, biomolecules data, and/or correlating data. The operational modules, information, and/or data may include the required information and/or may allow computing system, and specifically computing device, to perform the processes discussed herein for detecting cognitive diseases and impairments in humans.

Computing system, and specifically computing device of computing system, may also be in communication with external storage component. External storage component may be configured to store various modules, data and/or electronic information relating to various other aspects of computing system, similar to storage component of computing device(s). Additionally, external storage component may be configured to share (e.g., send and receive) data and/or electronic information with computing device(s) of computing system. In the non-limiting example shown in FIG. 8, external storage component may include any or all of the operational modules and/or data shown to be stored on storage component. Additionally, external storage component may also include a secondary database that user may interact with, provide information/data to, and/or may include information/data relating to poster. In a non-limiting example, external storage component may be a cloud-based storage component or system. In other non-limiting examples, external storage component may also include and/or be in communication with a neural network to aid in computation and/or data processing as discussed herein.

In a non-limiting example shown in FIG. 8, computing device(s) may be in communication with and/or may be configured to share (e.g., send and receive) data and/or electronic information over a network. Network may represent a closed network, such as a local area network (LAN) or may include the internet. Network may also include secondary database including similar data as storage component, and/or may include or be in communication with a neural network to aid in computation and/or data processing as discussed herein.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As discussed herein, various systems and components are described as “obtaining” data. It is understood that the corresponding data can be obtained using any solution. For example, the corresponding system/component can generate and/or be used to generate the data, retrieve the data from one or more data stores (e.g., a database), receive the data from another system/component, and/or the like. When the data is not generated by the particular system/component, it is understood that another system/component can be implemented apart from the system/component shown, which generates the data and provides it to the system/component and/or stores the data for access by the system/component.

The foregoing drawings show some of the processing associated according to several embodiments of this disclosure. In this regard, each drawing or block within a flow diagram of the drawings represents a process associated with embodiments of the method described. It should also be noted that in some alternative implementations, the acts noted in the drawings or blocks may occur out of the order noted in the figure or, for example, may in fact be executed substantially concurrently or in the reverse order, depending upon the act involved. Also, one of ordinary skill in the art will recognize that additional blocks that describe the processing may be added.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise. “Approximately” as applied to a particular value of a range applies to both values, and unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. A method for detecting a cognitive disease, the method comprising:
providing a saliva sample from a human subject;
subjecting at least a portion of the saliva sample to a spectroscopic analysis to produce a sample spectroscopic signature for the saliva sample;
analyzing the produced sample spectroscopic signature using a predetermined statistical model, the predetermined statistical model based on spectroscopic signatures for a plurality modeling samples,
wherein the spectroscopic signatures for each of the plurality of modeling samples are associated with one of a plurality of predetermined cognitive categories; and
correlating the produced sample spectroscopic signature with one of the plurality of predetermined cognitive categories based on the spectroscopic signatures for each of the plurality of modeling samples of the predetermined statistical model.

2. The method of claim 1, where the plurality of predetermined cognitive categories include:
a cognitive healthy class;
an Alzheimer's disease class; and
a mild cognitive impairment class.

3. The method of claim 2, wherein the correlating of the produced sample spectroscopic signature further includes:
identifying the human subject as being associated with one of the cognitive healthy class, the Alzheimer's disease class, or the mild cognitive impairment class, and
detecting the cognitive disease in the human subject in response to identifying the human subject being associated with one of the Alzheimer's disease class or the mild cognitive impairment class.

4. The method of claim 1, wherein the subjecting of at least the portion of the saliva sample to the spectroscopic analysis further includes:
performing Raman spectroscopy on at least the portion of the saliva sample, the Raman spectroscopy selected from the group consisting of: near-infrared (NIR) Raman spectroscopy, Raman microspectroscopy, Surface Enhanced Raman spectroscopy (SERS), surface enhanced resonance Raman spectroscopy (SERRS), Raman hyper spectroscopy, Fourier transform Raman spectroscopy, and coherent anti-Stokes Raman Spectroscopy (CARS).

5. The method of claim 1, wherein the subjecting of at least the portion of the saliva sample to the spectroscopic analysis further includes:
exposing biomolecules of the saliva sample to a spectroscopic analysis, the biomolecules including at least one of structural properties, conformational properties, or compositional variations that define the produced sample spectroscopic signature for the saliva sample.

6. The method of claim 5, wherein the biomolecules include at least one of: proteins, lipids, peptides, amino acids, electrolytes, mucus, enzymes, or antibacterial species.

7. The method of claim 1, wherein the subjecting at least the portion of the saliva sample to the spectroscopic analysis further includes:
subjecting a plurality of portions of the saliva sample to the spectroscopic analysis to produce a plurality of distinct sample spectroscopic signatures for the saliva sample, each of the plurality of portions positionally distinct from the others in the saliva sample.

8. The method of claim 7, wherein:
the analyzing of the produced sample spectroscopic signature using the predetermined statistical model further includes:
analyzing each of the plurality of the produced sample spectroscopic signatures using the predetermined statistical model; and
the correlating of the produced sample spectroscopic signature with one of the plurality of predetermined cognitive categories further includes:
correlating each of the plurality of produced sample spectroscopic signatures with one of the plurality of predetermined cognitive categories based on the spectroscopic signatures for each of the plurality of modeling samples of the predetermined statistical model.

9. The method of claim 8, further comprising:
determining a final, predetermined cognitive category for the saliva sample based on each of the plurality of correlated, produced sample spectroscopic signatures.

10. The method of claim 1, further comprising:
discarding predetermined portions of the produced sample spectroscopic signature prior to the analyzing of the produced sample spectroscopic signature using the predetermined statistical model,
wherein the discarded predetermined portions of the produced sample spectroscopic signature are inconclusive for correlating the produced sample spectroscopic signature with one of the plurality of predetermined cognitive categories based on the spectroscopic signatures for each of the plurality of modeling samples of the predetermined statistical model.

11. The method of claim 1, wherein the produced sample spectroscopic signature for the saliva sample includes a vibrational signature of the provided saliva sample.

12. A system comprising:
a spectroscopy device subjecting at least a portion of a saliva sample from a human to a spectroscopic analysis to produce a sample spectroscopic signature for the saliva sample; and
at least one computing device in operable communication with the spectroscopy device, the at least one computing device configured to detect a cognitive disease in the human subject by:
analyzing the produced sample spectroscopic signature using a predetermined statistical model, the predetermined statistical model based on spectroscopic signatures for a plurality modeling samples,
wherein the spectroscopic signatures for each of the plurality of modeling samples are associated with one of a plurality of predetermined cognitive categories; and
correlating the produced sample spectroscopic signature with one of the plurality of predetermined cognitive categories based on the spectroscopic signatures for each of the plurality of modeling samples of the predetermined statistical model.

13. The system of claim 12, where the plurality of predetermined cognitive categories include:
a cognitive healthy class;
an Alzheimer's disease class; and
a mild cognitive impairment class.

14. The system of claim 13, wherein the at least one computing device correlates the produced sample spectroscopic signature further by:
identifying the human subject as being associated with one of the cognitive healthy class, the Alzheimer's disease class, or the mild cognitive impairment class, and
detecting the cognitive disease in the human subject in response to identifying the human subject being associated with one of the Alzheimer's disease class or the mild cognitive impairment class.

15. The system of claim 12, wherein the spectroscopy device subjects at least the portion of the saliva sample to the spectroscopic analysis by:
performing spectroscopy on at least the portion of the saliva sample, the spectroscopy selected from the group consisting of: near-infrared (NIR) Raman spectroscopy, Raman microspectroscopy, Surface Enhanced Raman spectroscopy (SERS), surface enhanced resonance Raman spectroscopy (SERRS), Raman hyper spectroscopy, Fourier transform Raman spectroscopy, IR absorption spectroscopy, Fourier Transform Infrared absorption (FTIR), Attenuated Total Reflection (ATR) FTIR, IR reflection spectroscopy, vibrational spectroscopy, and coherent anti-Stokes Raman Spectroscopy (CARS).

16. The system of claim 12, wherein the spectroscopy device subjects at least the portion of the saliva sample to the spectroscopic analysis by:
exposing biomolecules of the saliva sample to a spectroscopic analysis, the biomolecules including at least one of structural properties, conformational properties, or compositional variations that define the produced sample spectroscopic signature for the saliva sample, and
wherein the biomolecules include at least one of: proteins, lipids, peptides, amino acids, electrolytes, mucus, enzymes, or antibacterial species.

17. The system of claim 12, wherein the spectroscopy device subjects at least the portion of the saliva sample to the spectroscopic analysis by:
subjecting a plurality of portions of the saliva sample to the spectroscopic analysis to produce a plurality of distinct sample spectroscopic signatures for the saliva sample, each of the plurality of portions positionally distinct from the others in the saliva sample.

18. The system of claim 17, wherein the at least one computing device:
analyzes the produced sample spectroscopic signature using the predetermined statistical model by:
analyzing each of the plurality of the produced sample spectroscopic signatures using the predetermined statistical model; and
correlates the produced sample spectroscopic signature with one of the plurality of predetermined cognitive categories by:
correlating each of the plurality of produced sample spectroscopic signatures with one of the plurality of predetermined cognitive categories based on the spectroscopic signatures for each of the plurality of modeling samples of the predetermined statistical model.

19. The system of claim 18, wherein the at least one computing device configured to detect the cognitive disease in the human subject further by:
determining a final, predetermined cognitive category for the saliva sample based on each of the plurality of correlated, produced sample spectroscopic signatures.

20. The system of claim 12, wherein the at least one computing device configured to detect the cognitive disease in the human subject further by:
discarding predetermined portions of the produced sample spectroscopic signature prior to the analyzing of the produced sample spectroscopic signature using the predetermined statistical model,
wherein the discarded predetermined portions of the produced sample spectroscopic signature are inconclusive for correlating the produced sample spectroscopic signature with one of the plurality of predetermined cognitive categories based on the spectroscopic signatures for each of the plurality of modeling samples of the predetermined statistical model.
